(19) United States
(12) Patent Application Publication
(10) Pub. No.: US 2010/0273167 A1
(43) Pub. Date: Oct. 28, 2010

(54) METHODS FOR DETERMINING PRENATAL ALCOHOL EXPOSURE

(76) Inventors: Jennifer Hipp, Winston-Salem, NC (US); Jason D. Hipp, Bethesda, MD (US); Shay Soker, Greensboro, NC (US)

Correspondence Address: MYERS BIGEL SIBLEY & SAJOVEC, PO BOX 37428, RALEIGH, NC, 27627, US

(21) Appl. No.: 12/763,789

(22) Filed: Apr. 20, 2010

Related U.S. Application Data
(60) Provisional application No. 61/170,742, filed on Apr. 20, 2009.

(51) Int. Cl. C12Q 1/68; C07H 21/04

(52) U.S. Cl. 435/6; 536/23.2; 536/23.5

(57) ABSTRACT

Provided herein are methods for determining ethanol exposure of a prenatal subject, including measuring whether or not amniotic fluid stem cells collected from amniotic fluid surrounding the prenatal subject have a upregulation or expression of one or more genes of a first predetermined combination and/or a downregulation of expression of one or more genes of a second predetermined combination. Also provided are methods for determining ethanol exposure of a prenatal subject which methods include measuring alkaline phosphatase activity and/or calcium deposition of amniotic fluid stem cells collected from amniotic fluid surrounding the prenatal subject.

### RELATED APPLICATIONS

This application claims the benefit under 35 U.S.C. section 119(e) of U.S. Provisional Patent Application No. 61/170,742, filed Apr. 20, 2009, the disclosure of which is incorporated by reference herein in its entirety.

### GOVERNMENT SUPPORT

This invention was made with Government support under contract number NIAAA F30AA016446-02 from the National Institute of Alcohol Abuse and Alcoholism at the National Institutes of Health. The U.S. Government has certain rights to this invention.

### FIELD OF THE INVENTION

The present invention concerns methods for detecting prenatal alcohol exposure and cDNAs useful for carrying out such methods.

### BACKGROUND OF THE INVENTION

Maternal alcohol consumption during pregnancy commonly causes abnormal growth and morphogenesis of the fetus (Astley et al., Diagnosing the full spectrum of fetal alcohol-exposed individuals: introducing the 4-digit diagnostic code. Alcohol. 2000; 35:400-410). This spectrum of defects is known as Fetal Alcohol Spectrum Disorder (FASD; including Fetal Alcohol Syndrome) and occurs in approximately 0.03-0.15 percent of all live births in the United States (Centers for Disease Control and Prevention. Fetal alcohol syndrome—Alaska, Arizona, Colorado, and New York. 2002:433-435). FASD is associated with mild to moderate mental retardation to more severe outcomes that include growth deficiencies, craniofacial defects, and dysmorphogenesis of the brain (Habbick et al., Bone age and growth in fetal alcohol syndrome. Alcohol Clin Exp Res. 1998; 22:1312-1316; Lemoine et al., Children of alcoholic parents—observed anomalies: discussion of 127 cases. Ther Drug Monit. 2003; 25:132-136; Johnson et al., Fetal alcohol syndrome: craniofacial and central nervous system manifestations. Am J Med Genet. 1996; 61:329-339). Prenatal alcohol exposure can also result in multiple organ defects of the heart, eye, kidneys, muscle, skeleton (Randall et al., Ethanol-induced malformations in mice. Alcohol Clin Exp Res. 1977; 1:219-224; Becker et al., Teratogenic actions of ethanol in the mouse: a minireview. Pharmacol Biochem Behay. 1996; 55:501-513; Sulik et al. Fetal alcohol syndrome: embryogenesis in a mouse model. Science. 1981; 214:936-938; Parnell et al., Maternal oral intake mouse model for fetal alcohol spectrum disorders: ocular defects as a measure of effect. Alcohol Clin Exp Res. 2006; 30:1791-1798; Herrmann et al., Tetraectrodactyly and other skeletal manifestations in the fetal alcohol syndrome. Eur J. Pediatr. 1980; 133:221-226) and permanent growth retardation (Habbick et al., Bone age and growth in fetal alcohol syndrome. Alcohol Clin Exp Res. 1998; 22:1312-1316).

Ethanol is a teratogen because of its ability to persistently disrupt cell functions beyond a specific exposure period. Some stem cells are particularly sensitive to exposure (Hao et al., Human neural stem cells are more sensitive than astrocytes to ethanol exposure. Alcohol Clin Exp Res. 2003; 27:1310-1317). Stem cells have the potential to proliferate as non-comitted cells and differentiate into multiple lineages (Daley et al., Realistic prospects for stem cell therapeutics. Hematology Am Soc Hematol Educ Program. 2003; 398-418). Understanding how ethanol affects stem cells and their differentiation potential may provide insights into the mechanism underlying the genesis of FASD.

The in vivo and in vitro effects of ethanol may vary from induction of apoptosis to the inhibition of proliferation, differentiation, migration or other functions (Gong et al., Inhibitory effect of alcohol on osteogenic differentiation in human bone marrow-derived mesenchymal stem cells. Alcohol Clin Exp Res. 2004; 28:468-479; Li et al., Disruption of cell cycle kinetics and cyclin-dependent kinase system by ethanol in cultured cerebellar granule progenitors. Brain Res Dev Brain Res. 2001; 132:47-58; Miller et al., Intracellular recording and injection study of corticospinal neurons in the rat somatosensory cortex: effect of prenatal exposure to ethanol. J Comp Neurol. 1990; 297:91-105; Siegenthaler et al., Transforming growth factor beta1 modulates cell migration in rat cortex: effects of ethanol. Cereb Cortex. 2004; 14:791-802). Ethanol has been shown to affect membrane signaling pathways (Resnicoff et al., Ethanol inhibits the autophosphorylation of the insulin-like growth factor 1 (IGF-1) receptor and IGF-1-mediated proliferation of 3T3 cells. J Biol Chem. 1993; 268:21777-21782) and cell adhesion (Vangipuram et al., Ethanol increases fetal human neurosphere size and alters adhesion molecule gene expression. Alcohol Clin Exp Res. 2008; 32:339-347; Charness et al., Ethanol inhibits neural cell-cell adhesion. J Biol Chem. 1994; 269:9304-9309), generate free radicals (Chen et al., Free radicals and ethanol-induced cytotoxicity in neural crest cells. Alcohol Clin Exp Res. 1996; 20:1071-1076), and alter the binding of transcription factors (Pignataro et al., Alcohol regulates gene expression in neurons via activation of heat shock factor 1. J. Neurosci. 2007; 27:12957-12966). These cellular functions are also critical for stem cell differentiation (Inui et al., Effects of beta mercaptoethanol on the proliferation and differentiation of human osteoprogenitor cells. Cell Biol Int. 1997; 21:419-425). The overlap of cellular functions that are affected by ethanol and are involved in stem cell differentiation might indicate that ethanol may affect stem cell differentiation.

There is no cure for FASD. Prevention is certain only if maternal alcohol consumption is avoided during pregnancy. Currently, prenatal alcohol exposure can be determined only through the interview of the biological mother or other family members knowledgeable of the mother's alcohol use during the pregnancy.

### SUMMARY OF THE INVENTION

Provided herein are methods for determining ethanol exposure of a prenatal subject, including measuring whether or not amniotic fluid stem cells collected from amniotic fluid surrounding the prenatal subject have a two-fold or greater upregulation of expression of each gene of a first predetermined combination of genes as compared to expression of each gene of said first predetermined combination by control amniotic fluid stem cells or fibroblast cells, wherein said two-fold or greater upregulation of expression of each gene of said first predetermined combination indicates ethanol exposure of said prenatal subject. In some embodiments, the first predetermined combination of genes comprises one or more, or five or more, or 10 or more, or 20 or more genes selected from the listing in Table 2A.

In some embodiments, the methods also include measuring whether or not the amniotic fluid stem cells have a two-fold or greater down-regulation of expression of each gene of a second predetermined combination of genes as compared to expression of each gene of said second predetermined combination by control amniotic fluid stem cells or fibroblast cells, wherein said two-fold or greater downregulation of expression of each gene of said second predetermined combination indicates ethanol exposure of said prenatal subject. In some embodiments, the second predetermined combination of genes comprises one or more, five or more, or 10 or more genes selected from the listing in Table 2B.

In some embodiments, the prenatal subject is a human subject, and in some embodiments the amniotic fluid stem cells are collected between 8 and 22 weeks of gestation.

In some embodiments, the measuring includes nucleic acid amplification and/or microarray analysis.

Also provided is a combination consisting essentially of a plurality of cDNAs encoding at least five, at least 10, or at least 20 genes selected from the listing in Table 2A. In some embodiments, the cDNAs are immobilized on a substrate.

Further provided are methods for determining ethanol exposure of a prenatal subject including: measuring whether or not amniotic fluid stem cells collected from amniotic fluid surrounding the prenatal subject have a two-fold or greater upregulation of expression of secreted phosphoprotein 1 (osteopontin, bone sialoprotein I, early T-lymphocyte activation 1) as compared to expression of secreted phosphoprotein 1 (osteopontin, bone sialoprotein I, early T-lymphocyte activation 1) by control amniotic fluid stem cells or fibroblast cells, wherein said two-fold or greater upregulation of expression of secreted phosphoprotein 1 (osteopontin, bone sialoprotein I, early T-lymphocyte activation 1) indicates ethanol exposure of said prenatal subject.

In some embodiments, the prenatal subject is a human subject, and in some embodiments the amniotic fluid stem cells are collected between 8 and 22 weeks of gestation.

In some embodiments, the measuring includes nucleic acid amplification and/or microarray analysis.

Also provided are methods for determining ethanol exposure of a prenatal subject including: measuring whether or not amniotic fluid stem cells collected from amniotic fluid surrounding the prenatal subject have a two-fold or greater down-regulation of expression of each gene of a predetermined combination of genes as compared to expression of each gene of said predetermined combination by control amniotic fluid stem cells or fibroblast cells, wherein said two-fold or greater downregulation of expression of each gene of said predetermined combination indicates ethanol exposure of said prenatal subject. In some embodiments, the first predetermined combination of genes comprises one or more, or five or more, or 10 or more genes selected from the listing in Table 2B.

In some embodiments, the prenatal subject is a human subject, and in some embodiments the amniotic fluid stem cells are collected between 8 and 22 weeks of gestation.

In some embodiments, the measuring includes nucleic acid amplification and/or microarray analysis.

Also provided is a combination consisting essentially of a plurality of cDNAs encoding at least five or at least 10 genes selected from the listing in Table 2B. In some embodiments, the cDNAs are immobilized on a substrate.

Further provided are methods for determining ethanol exposure of a prenatal subject including: providing amniotic fluid stem cells collected from amniotic fluid surrounding the prenatal subject; differentiating said amniotic fluid stem cells in osteogenic medium; and measuring whether or not said amniotic fluid stem cells have an alkaline phosphatase activity above a threshold of 6,000 Units/L at day 8, 10, 11 or 12 of said differentiating, wherein said alkaline phosphatase activity is measured as Units/L=liberation of 1 mmol of PNP per minute at 37° C. incubation per liter, wherein alkaline phosphatase activity above a threshold of 6,000 Units/L at day 8, 10, 11 or 12 indicates ethanol exposure of said prenatal subject.

In some embodiments, the methods further include: measuring whether or not calcium deposition at day 23 after said differentiating is above a threshold of 155 μg/mL, wherein calcium deposition above a threshold of 155 μg/mL at day 23 of said differentiating indicates ethanol exposure of said prenatal subject.

In some embodiments, the prenatal subject is a human subject, and in some embodiments the amniotic fluid stem cells are collected between 8 and 22 weeks of gestation.

Also provided are methods for determining ethanol exposure of a prenatal subject including: providing amniotic fluid stem cells collected from amniotic fluid surrounding said prenatal subject; differentiating said amniotic fluid stem cells in osteogenic medium; and measuring whether or not calcium deposition at day 23 after said differentiating is above a threshold of 155 μg/mL, wherein calcium deposition above a threshold of 155 μg/mL at day 23 of said differentiating indicates ethanol exposure of said prenatal subject.

In some embodiments, the prenatal subject is a human subject, and in some embodiments the amniotic fluid stem cells are collected between 8 and 22 weeks of gestation.

### BRIEF DESCRIPTION OF THE DRAWINGS

FIG. 1. Effect of ethanol on cell proliferation and viability. AFSC were cultured with 0, 25, 50, 75, and 100 mM of ethanol. (A) Proliferation rate was determined by cell number counts after 48 hours of ethanol exposure and compared to no ethanol treatment. The results are expressed as percentages relative to cells without ethanol (dark grey bars). The values shown are the mean+/−SD (n=5) of three independent experiments (*, p<0.03 by student T-test). (B) The effect of ethanol on the percentage of non-viable cells was determined by 7-AAD and flow cytometry. Light grey bars indicate the percentage of non-viable cells in the presence of various concentrations of ethanol. The data shown represents the mean number of apoptotic cells in 10,000 events of two independent experiments with error bars of SDs and were not significant. (C) Ethanol concentration in the media was measured by spectrophotometry. Results represent the mean +/−SD from three replicates.

FIG. 2. Effect of ethanol on OPN expression in AFSC. (A) Real-time RT PCR analysis of AFSC exposed to ethanol for 24 or 48 hours in growth media. CT values were determined from 3 independent experiments from two cell lines. ACT values were obtained by subtracting the CT values of β-actin. Mean fold change was determined by averaging the fold change from four independent experiments' pairs of control and ethanol-exposed AFSC. Black columns indicate AFSC without ethanol while grey columns indicate AFSC exposed to 100 mM ethanol. P-values were determined using a one-tailed paired t test with significance at 24 hours (P<0.020, n=3) and 48 hours (P<0.036, n=5). (B) Real-time PCR analysis of AFSC exposed to ethanol for 24 hours in osteogenic media. CT values were determined from 3 independent experiments using two cell lines. ACT values were obtained by subtracting the CT values of β-actin. Mean fold change was determined by averaging the fold change from 3 independent experiments' pairs of control and ethanol-exposed AFSC. P-values were determined using a one-tailed paired t test with significance at 24 (P<0.018).

FIG. 3. Effect of ethanol on alkaline phosphatase activity in AFSC upon osteogenic differentiation. AFSC were cultured with or without ethanol exposure for the first 48 hours of osteogenic differentiation. Ethanol was removed and AFSC continued to differentiate until days 7-10 and assess for alkaline phosphatase activity. Alkaline phosphatase activity was determined by spectrophotometric measurement of p-nitrophenol conversion. AFSC exposed to ethanol showed a modest yet significant increase in ALP activity at day 9 and 10 of osteogenic differentiation (*, p<0.001; ANOVA and two-tail T-test). The values shown are the mean +/−SD from at least ten replicate cultures and similar patterns were observed in a different cell line. Difference between control and treated cultures were evaluated by t-test. Open squares, osteogenic media+EtOH (48 hours); black diamonds, osteogenic media; black circles, growth media.

FIG. 4. Alkaline phosphatase activity when exposed to ethanol at midpoint of osteogenic differentiation. AFSC were treated with 100 mM ethanol at day 8 of osteogenic differentiation for 48 hours. Black columns indicate AFSC that were not exposed to ethanol while grey bars indicate AFSC that were exposed to ethanol. Ethanol exposure during midpoint of differentiation had no significant effect on alkaline phosphatase activity (t test p<0.42). The values shown are the mean +/−SD of twenty cultures.

FIG. 5. The effect of ethanol on calcium deposition upon osteogenic differentiation. AFSC were exposed to 100 mM ethanol during the first 48 hours of osteogenic differentiation. At day 23 of differentiation, AFSC were stained for calcium deposition by alizarin red staining. AFSC exposed to 48 hours of 100 mM ethanol significantly increased calcium deposition when compared to controls (155.1±75.8 μg/mL versus 77.4±26.9 μg/mL). Calcium deposition was detected in non-differentiated AFSC at a basal level of 53.3±2.2 μg/mL. The values shown are the mean +/−SD (n=3-6) and similar results were confirmed in another cell line. * One-tailed t-test of significance of p<0.006.

### DETAILED DESCRIPTION OF THE PREFERRED EMBODIMENTS

Herein are provided methods and cDNAs useful in determining the presence or absence of exposure of prenatal subjects to alcohol. In preferred embodiments, amniotic fluid stem cells (AFSC) are used to determine the presence or absence of one or more positive or negative markers and/or indicators of prenatal alcohol exposure.

The disclosures of all cited United States Patent references are hereby incorporated by reference to the extent that they are consistent with the disclosures herein. As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” and “/” refer to and encompass any and all possible combinations of one or more of the associated listed items, as well as the lack of combinations when interpreted in the alternative (“or”).

“Prenatal” subjects as used herein refer to embryonic or fetal subjects, including, but not limited to, human prenatal subjects.

Amniotic fluid stem cells (AFSC) are cells of embryonic or fetal origin that have been previously described (U.S. Patent Application Publication No. 2005/0124003 to Atala et al.; De Coppi et al. (2007) Nature Biotechnology 25(1):100-6). AFSC are pluripotent stem cells with extensive self-renewal potential and are capable of differentiating in vitro into bone (osteogenic differentiation), muscle (myogenic differentiation), fat (adipogenic differentiation), endothelium (endothelial differentiation), liver (hepatic differentiation), and neuron-like cells (neurogenic differentiation). Preferably, the AFSC are also characterized by the ability to be grown in vitro without the need for feeder cells.

However, and as described more fully below, AFSC which have been exposed to alcohol (e.g., ethanol), as opposed to AFSC not exposed to alcohol, may be predisposed to and/or induced/differentiated towards a particular lineage (e.g., osteogenic), and thus have impaired ability to differentiate into certain of the lineages listed above.

Though AFSC may be predisposed towards a particular lineage upon alcohol exposure, such exposure does not appear to affect c-kit expression (based upon in vitro alcohol exposure of AFSC). Therefore, AFSC collection making use of c-kit selection from tissues and/or amniotic fluid is not expected to be affected by prenatal alcohol exposure.

“Exposure” of cells to alcohol (e.g., ethanol) as used herein refers to the contact of the cells with alcohol in sufficient quantity (measured by, e.g., concentration) and time to elicit a differential expression response thereto, e.g., the positive or increased expression of one or more markers associated with differentiation into an osteogenic lineage; and/or negative or decreased expression of one or more markers associated with stem cells as described hereinbelow.

“Cells” used in carrying out the present invention are, in general, animal cells, including but not limited to human and non-human cells such as primate (e.g., monkey, chimpanzee, baboon), dog, cat, mouse, rat, horse, cow, pig, rabbit and goat cells, as well as avian, reptile and amphibian cells (e.g., chicken, turkey, duck, geese, quail, pheasant, frog, toad, etc.).

“Stem cell” as used herein refers to a cell that has the ability to replicate through numerous population doublings (e.g., at least 60-80), in some cases essentially indefinitely, and to differentiate into multiple cell lineages.

“Pluripotent” as used herein refers to a cell that can differentiate, upon appropriate stimulation, into each of osteogenic, adipogenic, myogenic, neurogenic, hematopoietic, and endothelial cells. A pluripotent cell can be self-renewing, and can remain dormant or quiescent with a tissue. Unlike a totipotent cell (e.g., a fertilized, diploid egg cell), however, a pluripotent cell cannot form a new blastocyst.

“Multipotent cell” as used herein refers to a cell that has the capacity to grow into any of a subset (2, 3, 4 or 5) of the corresponding animal cell types. However, unlike a pluripotent cell, a multipotent cell does not have the capacity to form all six of the cell types of the corresponding animal listed above.

“Expression” of a gene (e.g., encoding a specific marker) means that the gene is transcribed, and optionally, translated. Typically, expression of a gene encoding a specific marker will result in production of an encoded polypeptide. Gene expression may be measured by techniques known to those of skill in the art, e.g., microarray analysis, quantitative per, Southern, northern or western blot analysis, etc.

“Differential expression” refers to an increased, up-regulated or present (positive), or decreased, down-regulated or absent (negative), gene expression as detected by the absence, presence, or a Bayesian statistic (greater than 0), which corresponds to a significant difference in the amount of transcribed messenger RNA, translated protein, or other marker, in a sample.

“Isolated” as used herein signifies that the cells are placed into conditions other than their natural environment. The term “isolated” does not preclude the use of these cells thereafter in combinations or mixtures with other cells.

In general, AFSC are cells, or progeny of cells, that are found in or collected primarily from mammalian amniotic fluid, but may also be collected from mammalian chorionic villus or mammalian placental tissue. Human AFSC can be isolated from amniotic fluid between 8 and 22, or 10 and 20, or 14 and 18 weeks of gestation, and comprise approximately 1% of the cells present in amniotic fluid. In some embodiments, the cells are collected during the first or second trimester of gestation, e.g., during procedures to collect the fluid/tissue for prenatal genetic testing. For example, fluid may be collected by amniocentesis, in which amniotic fluid is collected for testing (typically performed between weeks 15 and 20 of gestation). Tissue may be collected by chorionic villus sampling (CVS) (typically performed between weeks 10 and 12).

In general, the tissue or fluid can be withdrawn by amniocentesis, punch-biopsy, homogenizing the placenta or a portion thereof, or other tissue sampling techniques, in accordance with known techniques. From the sample, stem cells or pluripotent cells may be isolated with the use of a particular marker or selection antibody that specifically binds stem cells, in accordance with known techniques such as affinity binding and/or cell sorting. Particularly suitable is the c-Kit antibody, which specifically binds to the c-kit receptor protein. C-kit antibodies are known (see, e.g., U.S. Pat. Nos. 6,403,559, 6,001,803, and 5,545,533). A preferred antibody is c-Kit (CD117) monoclonal IgG that recognizes an epitope corresponding to amino acids 23-322 mapping near the human c-kit N-terminus. CD117 antibodies are available from Santa Cruz Biotechnology, Inc., 2145 Delaware Avenue, Santa Cruz, Calif., USA 95060, under catalog number SC-17806. In other embodiments, cells are c-kit selected with monoclonal andi-CD117 directly conjugated to MicroBeads (Miltenyi Biotec).

In some embodiments, AFSC used to carry out the present invention are “pluripotent.” Hence, they differentiate, upon appropriate stimulation, into at least osteogenic, adipogenic, myogenic, endothelial, neurogenic, and hepatic cells. Appropriate stimulation, for example, may be as follows. Osteogenic induction: Seed c-Kit+ cells at a density of 3,000 cells/cm² and culture in DMEM low glucose medium with 10% FBS (Gibco/BRL), antibiotics (Pen/Strep, Gibco/BRL) and osteogenic supplements (100 nM dexamethasone (Sigma-Aldrich), 10 mM beta-glycerophosphate (Sigma-Aldrich) and 0.05 mM ascorbic acid-2-phosphate (Wako Chemicals, Irving, Tex.). Adipogenic induction: Seed c-Kit+ cells at a density of 3000 cells/cm² and culture in DMEM low glucose medium with 10% FBS, antibiotics (Pen/Strep, Gibco/BRL) and adipogenic supplements (1 μM dexamethasone, 1 mM 3-isobutyl-1-methylxantine, 10 μg/ml insulin, and 60 μM indomethacin (all from Sigma-Aldrich)). Myogenic induction: Seed c-Kit+ cells at a density of 3,000 cells/cm² onto Matrigel-precoated plastic plates (Collaborative Biomedical Products, incubation for 1 h at 37° C. at 1 mg/ml in DMEM) and culture in DMEM low-glucose formulation supplemented with 10% horse serum (Gibco/BRL), 0.5% chick embryo extract (Gibco/BRL), and Pen/Strep. Twelve hours after seeding, add 3 μM 5-aza-2′-deoxycytidine (5-azaC, Sibma-Aldrich) to the medium and incubate for 24 h. Thereafter, continue incubation in complete medium lacking 5-azaC, with medium changes every 3 days. Endothelial induction: Seed c-Kit+ cells at a density of 3,000 cells/cm² onto plastic plates pre-coated with gelatin. Maintain in culture for 1 month in endothelial cell medium-2 (EG-M™-2, Clonetics, Cambrex Bioproducts) supplemented with 10% FBS and Pen/Strep. Add recombinant human bFGF (StemCell Technologies) at intervals of 2 d at 2 ng/ml. Neurogenic induction: Seed c-Kit+ cells at a density of 3,000 cells/cm² onto tissue culture plastic plates and culture in DMEM low-glucose medium, Pen/Strep, supplemented with 2% DMSO, 200 butylated hydroxyanisole (BHA, Sigma-Aldrich) and NGF (25 ng/ml). After 2 d, return cells to AFS growth medium lacking DMSA and BHA but still containing NGF. Add fresh NGF at intervals of 2 d. Hepatic induction: Seed c-Kit+ cells at a density of 5,000 cells/cm² onto Matrigel-precoated plastic plates. Expand in AFS growth medium for 3 d until semi-confluent. Change medium to DMEM low-glucose formulation containing 15% FBS, 300 μM monothioglycerol (Sigma-Aldrich), 20 ng/ml hepatocyte growth factor (Sigma-Aldrich), 10 ng/ml oncostatin M (Sigma-Aldrich), 10⁻⁷ M dexamethasone (Sigma-Aldrich), 100 ng/ml FGF4 (Peprotech), 1× ITS (insulin, transferrin, selenium; Roche) and Pen/Strep. Maintain cells in this differentiation medium for 2 weeks, with medium changes every third day. Harvest using trypsin and plate into a collagen sandwich gel (0.11 mg/cm² for both the lower and upper layers).

In preferred embodiments, no feeder layer or leukaemia inhibitory factor (LIF) are required either for expansion or maintenance of AFSC in the entire culture process. Also, in some embodiments, AFSC can proliferate through at least 60 or 80 population doublings or more when grown in vitro. In preferred embodiments, AFSC can proliferate through 100, 200 or 300 population doublings or more when grown in vitro. In vitro growth conditions for such determinations may be: (a) placing of the amniotic fluid or other crude cell-containing fraction from the mammalian source onto a 24 well Petri dish a culture medium [α-MEM (Gibco) containing 15% ES-FBS, 1% glutamine and 1% Pen/Strept from Gibco supplemented with 18% Chang B and 2% Chang C from Irvine Scientific], upon which the cells are grown to the confluence, (b) dissociating the cells by 0.05% trypsin/EDTA (Gibco), (c) isolating an AFSC subpopulation based on expression of a cell marker c-Kit using mini-MACS (Mitenyl Biotec Inc.), (d) plating of cells onto a Petri dish at a density of 3-8×10³/cm², and (e) maintaining the cells in culture medium for more than the desired time or number of population doublings.

AFSC used to carry out the present invention are preferably positive for alkaline phosphatase, preferably positive for Thy-1, and preferably positive for Oct4, all of which are known markers for embryonic stem cells, and all of which can be detected in accordance with known techniques. See, e.g., Rossant, J., Stem cells from the Mammalian blastocyst. Stem Cells, 2001. 19(6): p. 477-82; Prusa, A. R., et al., Oct-4-expressing cells in human amniotic fluid: a new source for stem cell research? Hum Reprod, 2003. 18(7): p. 1489-93. In addition, AFSC are preferably negative for CD34.

In a particularly preferred embodiment, the AFSC do not form a teratoma when undifferentiated AFSC are grown in vivo. For example, undifferentiated AFSC do not form a teratoma within one or two months after intraarterial injection into a 6-8 week old mouse at a dose of 5×10⁶ cells per mouse.

### Detection of Alcohol Exposure.

In some embodiments, detection of exposure of AFSC to alcohol is carried out by the detection of markers of osteogenic differentiation, wherein upregulation of expression of at least one osteogenic specific gene indicates differentiation of said cell into an osteogenic specific cell line. In some embodiments, detection includes measurement of expression of osteopontin (OPN, or secreted phosphoprotein 1 (SPP1)). Osteopontin is an extracellular structural protein normally found in bone. In some embodiments, osteogenic specific genes include: intracellular adhesion molecule 1 (ICAM1), osteomodulin (OMD), tissue inhibitor of metalloproteinase 4 (TIMP4), sex determining region Y box 4 (SOX4), crystallin alpha B (CRYAB), secreted phosphoprotein 1 (SPP1), v-fos FBJ murine steosarcoma viral oncogene homolog (FOS), alpha V integrin (ITGAV), prolactin (PRL), alpha 4 integrin (ITGA4), peroxisome proliferative activated receptor gamma (PPARG), secreted protein, acidic, cystein-rich (SPARC), sarcoma amplified sequence (SAS), and bone morphogenetic protein 1 (BMP1). See U.S. Patent Application No. 2006/0246488 to Hipp et al.

In some embodiments, detection of exposure of AFSC to alcohol is carried out by detection of upregulated and/or downregulated genes of one or more gene ontologies/molecular functions, as compared to a predetermined expression level for AFSC not exposed to alcohol or as compared to a control cell line. Control AFSC lines may be prepared from a non-ethanol exposed donor or an established non-ethanol exposed AFSC line. Other control cells and comparisons thereto may be determined through routine testing by those of skill in the art.

Upregulation (e.g., by 2-fold or more) of genes in one, two, three, four or five or more of the following gene ontology categories may indicate alcohol exposure to AFSC: biomineral & ossification, organ morphogenesis, organ development, reproductive developmental process, skeletal development, system development, blood vessel morphogenesis, tyrosine kinase signaling pathway, IGF receptor signaling pathway, and developmental process.

For example, according to some embodiments upregulation of one or more, or at least 5, 10, 15, 20, 25, 30, 35, 40, 45, 50, 55 or 60, of the following genes (e.g., by 2-fold or more) indicates alcohol exposure: signal sequence receptor gamma, lumican, solute carrier family 7 (cationic amino acid transporter, y+ system), member 8, BCL2-associated X protein, regulator of G-protein signaling 2 24 kDa, calreticulin, ectonucleotide pyrophosphatase/phosphodiesterase 1, endothelin receptor type A, ring finger protein 128, chromosome 1 open reading frame 54, collagen type III alpha 1 (Ehlers-Danlos syndrome type IV, autosomal dominant), transducin (beta)-like 1×-linked, BCL2-associated X protein, secreted phosphoprotein 1 (osteopontin, bone sialoprotein I, early T-lymphocyte activation 1), nuclear receptor subfamily 2 group F member 2, SRY (sex determining region Y)-box 4, SRY (sex determining region Y)-box 11, collagen type III alpha 1 (Ehlers-Danlos syndrome type IV, autosomal dominant), ATPase, H+ transporting lysosomal 70 kDa V1 subunit A, cornichon homolog 3, DEAD (Asp-Glu-Ala-Asp) box polypeptide 17, malignant fibrous histiocytoma amplified sequence 1, UDP-N-acetyl-alpha-D-galactosamine:polypeptide N-acetylgalactosaminyltransferase 10 (GalNAc-T10), chromosome 1 open reading frame 121, RNA binding motif protein 25, phospholipase A2 group IVA (cytosolic, calcium-dependent), sphingomyelin phosphodiesterase acid-like 3A, SKI-like, KIAA1033, MADS box transcription enhancer factor 2 polypeptide C (myocyte enhancer factor 2C), ets variant gene 1, PTPRF interacting protein binding protein 1 (liprin beta 1), GTP binding protein overexpressed in skeletal muscle, ATPase, H+ transporting lysosomal 9 kDa V0 subunit e, SEC24 related gene family, member D, plasminogen activator urokinase, chromosome 1 open reading frame 139, secreted protein acidic cysteine-rich (osteonectin), SRY (sex determining region Y)-box 11, forkhead box F1, phosphoinositide-3-kinase, regulatory subunit 1 (p85 alpha), adaptor-related protein complex 1 sigma 1 subunit, insulin-like growth factor 1 receptor, transmembrane protein 35, iduronate 2-sulfatase (Hunter syndrome), oxidation resistance 1, cyclin G2, degenerative spermatocyte homolog 1 lipid desaturase, ATPase Ca++ transporting plasma membrane 1, steroid-5-alpha-reductase alpha polypeptide 1 (3-oxo-5 alpha-steroid delta 4-dehydrogenase alpha 1), glycosyltransferase 8 domain containing 1, ADP-ribosylation factor-like 7, calumenin, low density lipoprotein-related protein 12, matrix metallopeptidase 14, and 3-hydroxyisobutyryl-Coenzyme A hydrolase.

Similarly, down-regulation (e.g., by 2-fold or more) of genes in one, two, three, four or five or more of the following gene ontology categories indicates alcohol exposure to AFSC: negative regulation of cellular process, circulatory system process, glucose transport, embryonic development, multi-organism process, anatomical structure development, and regulation of biological quality.

For example, down-regulation of one or more, or at least 5, 10 or 15 of the following genes (e.g., by 2-fold or more) indicates alcohol exposure according to some embodiments: H2A histone family member X, ubiquitin-conjugating enzyme E2I, heterogeneous nuclear ribonucleoprotein A3, dickkopf homolog 1, glutathione peroxidase 3, endothelin 1 pentraxin-related gene, ankyrin repeat domain 1, TSPY-like 4, fibroblast growth factor 2 (basic), microfibrillar associated protein 5, heterogeneous nuclear ribonucleoprotein H1 (H), deleted in liver cancer 1, ADAM metallopeptidase with thrombospondin type 1 motif, 1, oxytocin receptor, neuregulin 1

In some embodiments, detection of exposure of AFSC to alcohol is carried out by measuring alkaline phosphatase activity using methods known in the art (e.g., by spectrophotometric measurement of p-nitrophenol conversion), and optional comparison to alkaline phosphatase activity level of AFSC which have not been exposed to alcohol.

In some embodiments, upon in vitro osteogenic induction, AFSC exposed to ethanol have increased alkaline phosphatase activity (e.g., at day 7, 8, 9, 10, 11, 12, 13, and/or 14 of osteogenic induction). For example, for AFSC seeded at a density of 6,468 cells/cm², an alkaline phosphatase activity between 6,400 Units/L and 6,800 Units/L, or between 6,500 Units/L and 6,700 Units/L, or between 6,550 Units/L and 6,650 Units/L (e.g., 6,600 Units/L), at day 8, 10 or 12 of osteogenic differentiation indicates alcohol exposure, wherein alkaline phosphatase activity in Units/L=liberation of 1 mmol of PNP per minute at 37° C. incubation per liter.

In some embodiments, for AFSC seeded at a density of 6,468 cells/cm², an alkaline phosphatase activity above a threshold of 6,000 Units/L, or above a threshold of 6,200 Units/L, or above a threshold of 6,400 Units/L, at day 8, 10 or 12 of osteogenic differentiation indicates alcohol exposure, wherein alkaline phosphatase activity in Units/L=liberation of 1 mmol of PNP per minute at 37° C. incubation per liter.

In some embodiments, detection of exposure of AFSC to alcohol may be carried out by measuring calcium deposition using methods known in the art (e.g., by alizarin red staining), and optional comparison to calcium deposition in AFSC which have not been exposed to alcohol.

In some embodiments, upon in vitro osteogenic induction, AFSC exposed to ethanol have increased calcium deposition at day 23 of differentiation. For example, for AFSC seeded at a density of 6,468 cells/cm², calcium deposition of between 100 and 300 or between 115 and 200 μg/ml, or between 125 and 175 μg/ml (e.g., 155 μg/ml) indicates alcohol exposure, while calcium deposition of between 50 μg/ml and 99 μg/ml, or between 60 μg/ml and 90 μg/ml, or between 70 μg/ml and 85 μg/ml (e.g., 77 μg/ml) does not indicate alcohol exposure, at day 23 of in vitro osteogenic differentiation.

In some embodiments, for AFSC seeded at a density of 6,468 cells/cm², calcium deposition above a threshold of 100 μg/ml, or above a threshold of 120 μg/ml, or above a threshold of 140 μg/ml, or above a threshold of 155 μg/ml, indicates alcohol exposure at day 23 of in vitro osteogenic differentiation.

One or a combination of two or more of the tests described herein may be used in an overall profile to determine whether prenatal alcohol exposure of a subject has occurred.

Applications of the methods and techniques described herein are also useful for evaluating stem cell differentiation; defining specific genetic signatures associated with prenatal alcohol exposure, etc.

cDNAs and their Uses.

cdNAs can be prepared by a variety of synthetic or enzymatic methods well known in the art. cDNAs can be synthesized, in whole or in part, using chemical methods well known in the art (Caruthers et al. (1980) Nucleic Acids Symp. Ser. (7)215-233). Alternatively, cDNAs can be produced enzymatically or recombinantly, by in vitro or in vivo transcription. See, e.g., U.S. Pat. No. 6,544,742 (Incyte).

Nucleotide analogs can be incorporated into cDNAs by methods well known in the art. Preferably, the incorporated analog will base pair with native purines or pyrimidines. For example, 2,6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically.

cDNAs can be synthesized on a substrate according to methods known in the art. Synthesis on the surface of a substrate may be accomplished using a chemical coupling procedure and a piezoelectric printing apparatus as described by Baldeschweiler et al. (PCT publication WO95/251116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means as known in the art, such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means. If desired, cDNAs may be bound to the substrate through a linker group, which are typically about 6 to 50 atoms long, to enhance exposure of the attached cDNA. Preferred linker groups include ethylene glycol oligomers, diamines, diacids and the like. In some embodiments, reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

A cDNA may represent the complete coding region of an mRNA or be designed or derived from unique regions of the mRNA or genomic molecule, an intron, a 5′ or 3′ untranslated region, or from a conserved motif. The cDNA is normally at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate cDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide may be used to detect a polynucleotide or cDNA in a sample using PCR.

The cDNAs of the invention can be incorporated, as lineage-specific groups thereof, into kits for the detection of lineage-specific differentiation (e.g., osteogenic differentiation), or de-differentiation, as described in U.S. Pat. No. 6,489,455 to Chenchik et al. (Clontech) and U.S. Pat. No. 5,994,076 to Chenchik et al. (Clontech).

As used herein, a combination “consisting essentially” of a plurality of cDNAs encoding one or more specific genes refers to a combination in which at least 50, 60, 70, 80, 90, 95, or 99% or more of the cDNAs encode one or more of the specific genes described herein to be detected for determination of ethanol exposure of the prenatal subject.

### Detection of Gene Expression.

Detection of the differential expression (including upregulation and downregulation of expression) of a gene or nucleic acid is known and can be carried out in accordance with known techniques (e.g., utilizing cDNAs as described herein), or variations thereof apparent to persons skilled in the art in view of the instant disclosure. See, e.g., U.S. Pat. Nos. 6,727,006; 6,682,888; 6,673,549; 6,673,545; 6,500,642; 6,489,455.

For example, the combinations of the invention may be used on an array. When the cDNAs of the invention are employed on a microarray, the cDNAs are arranged in an ordered fashion so that each cDNA is present at a specified location. Because the cDNAs are at specified locations on the substrate, the hybridization patterns and intensities, which together create a unique expression profile, can be interpreted in terms of expression levels of particular genes and can be correlated with or used to identify differentiation and/or de-differentiation as described herein.

The cDNAs or fragments or complements thereof may be used in various hybridization technologies, e.g., to detect differential expression of genes as described herein in cells as described herein. The cDNAs may be labeled using a variety of reporter molecules by either PCR, recombinant, or enzymatic techniques. For example, a commercially available vector containing the cDNA is transcribed in the presence of an appropriate polymerase, such as T7 or SP6 polymerase, and at least one labeled nucleotide. Commercial kits are available for labeling and cleanup of such cDNAs. Radioactive (Amersham Pharmacia Biotech (APB), Piscataway N.J.), fluorescent (Operon Technologies, Alameda Calif.), and chemiluminescent labeling (Promega, Madison Wis.) are well known in the art.

As known in the art, the stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5× saline sodium citrate (SSC) with 1% sodium dodecyl sulfate (SDS) at 60° C., that permit the formation of a hybridization complex between nucleic acid sequences that contain some mismatches. Subsequent washes are performed with buffers such as 0.2×SSC with 0.1% SDS at either 45° C. (medium stringency) or 65-68° C. (high stringency). At high stringency, hybridization complexes will remain stable only where the nucleic acid molecules are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

The present invention is explained in greater detail in the following non-limiting Examples.

### EXAMPLES

### Example 1

### Microarray Analysis of AFSC Exposed to Physiologically Relevant Level of Ethanol

Global gene expression analysis was performed to identify relationships between alcohol exposure and induction of genes related to stem cell differentiation in amniotic fluid stem cells (AFSC).

Human AFSC were seeded at 3,000 cells/cm² and maintained in culture as described previously (De Coppi et al., Isolation of amniotic stem cell lines with potential for therapy. Nat Biotechnol. 2007; 25:100-106). AFSC were grown in α-MEM medium (Gibco, Invitrogen) containing 15% ES-FBS, 1% glutamine, and 1% penicillin/streptomycin (Gibco), supplemented with 18% Chang B and 2% Chang C (Irvine Scientific) at 37° C. with 5% CO2 atmosphere. For dose-dependent studies on cell growth and viability, AFSC were treated with 25 mM, 50 mM, 75 mM, and 100 mM ethanol (Sigma-Aldrich, St. Louis, Mo.), and sealed with parafilm with media being replaced every 24 hours.

Ethanol concentrations used in these experiments are equivalent to the concentrations of alcohol in the blood achieved by social drinkers to chronic alcoholics (Adachi et al., Degrees of alcohol intoxication in 117 hospitalized cases. J Stud Alcohol. 1991; 52:448-453; Perper J A, Twerski A, Wienand J W. Tolerance at high blood alcohol concentrations: a study of 110 cases and review of the literature. J Forensic Sci. 1986; 31:212-221) and have been previously used in other in vitro experiments (Chen et al., Free radicals and ethanol-induced cytotoxicity in neural crest cells. Alcohol Clin Exp Res. 1996; 20:1071-1076). The duration of ethanol exposure (48 hr) was determined to induce the maximum effect without causing toxicity. In addition, a study on the disposition of ethanol in the amniotic fluid and maternal blood in early second trimester females showed a delay in the clearance of ethanol from the amniotic fluid (Brien et al., 1983). This suggests that ethanol serves as a reservoir ethanol and that the duration of exposure of ethanol to the fetus may be longer than previously thought. Ethanol concentration was measured spectrophotometrically using Ethanol L3K assay (Diagnostic Chemicals Limited, Oxford Conn.) according to the manufacturer's protocol.

Forty-eight hours after seeding, ethanol was added to the media and parafilm sealed to prevent evaporation. Control groups were not treated with ethanol but otherwise treated identically. Proliferation rate was determined by measuring the number of AFSC after 48 hours of various ethanol concentrations. Cell number was measured using a Coulter counter. Cell viability was determined by propidium iodine (PI) exclusion. AFSC were dissociated by 0.05% trypsin/EDTA (Gibco), centrifuged at 1,500 RPM for 5 minutes, and resuspended in 1 ml of PBS in 15 ml polypropylene tubes. 50 μl of PI, a nucleotide analogue, was added to each tube and incubated for 1 hour at room temperature. Cells were centrifuged, supernatant removed, and washed twice with a wash buffer. Samples were analyzed by flow cytometry using the FL-1 channel.

Total RNA was isolated from AFSC using PerfectPure RNA Cultured Cell Kit (5 Prime) in accordance with the manufacturer's protocol. Following the procedure, DNA digestion was included as recommended by the supplier to eliminate the contamination of genomic DNA. Quality of total RNA was assessed by the spectrophotometric ratio of 260/280. For the reverse-transcriptase reaction, SuperscriptII reverse transcription reagents (Invitrogen) were used. Briefly, 1 μg of RNA was converted to cDNA. PCR amplification was performed with TaqMan Universal Master Mix (Applied Biosystems). Reactions were performed in duplicate, containing 1 μl of cDNA, 1.25 μl probe, 12 μl Master Mix, 10 μl DI H₂0 and were analyzed in a 96-well optical reaction plate (Applied Biosystems). Reactions were amplified and quantified using an ABI 7700 sequence detectors and manufacturer's software (Applied Biosystems). On demand fluorescent probes for the following genes: β-actin and osteopontin. The threshold cycle (Ct) indicates the fractional cycle number at which the amount of amplified target reaches a defined threshold. ΔCt was obtained by subtracting the Ct values of endogenous controls (β-actin) from the Ct values of the target genes.

Microarray Analyses. Two microarray analyses were performed on AFSC sealed with parafilm for 48 hours with or without 100 mM of ethanol. Fragmented antisense cRNA was used for hybridizing to human U133 A arrays (Affymetrix, Inc. Santa Clara, Calif., USA) at the Core Genomic Facility of Wake Forest University School of Medicine. These data are deposited in NCBI's Gene Expression Omnibus (GEO) and are accessible through GEO series accession numbers GSE13569, in accordance with MIAME standards.

Raw CEL files were provided by the Microarray Core Facility of the Wake Forest University School of Medicine and were then analyzed with a software package AffylmGUI (Affymetrix LIMMA, Linear Models for Microarray Data, Graphical User Interfaces) (Wettenhall et al., limmaGUI: a graphical user interface for linear modeling of microarray data. Bioinformatics. 2004; 20:3705-3706; Wettenhall et al., affylmGUI: a graphical user interface for linear modeling of single channel microarray data. Bioinformatics. 2006; 22:897-899). Within AffylmGUI, gene expression values were summarized with RMA. RMA adjusts for background noise, performs a quantile normalization, transforms the data into log base 2, and then summarizes the multiple probes into one intensity (Bolstad et al., A comparison of normalization methods for high density oligonucleotide array data based on variance and bias. Bioinformatics. 2003; 19:185-193; Irizarry et al., Exploration, normalization, and summaries of high density oligonucleotide array probe level data. Biostatistics. 2003; 4:249-264; Irizarry et al., Summaries of Affymetrix GeneChip probe level data. Nucleic Acids Res. 2003; 31:e15). Quantification of relative differences in gene expression among the groups of interest was accomplished using AffylmGUI, the sister package of limmaGUI (Wettenhall et al., limmaGUI: a graphical user interface for linear modeling of microarray data. Bioinformatics. 2004; 20:3705-3706; Wettenhall et al., affylmGUI: a graphical user interface for linear modeling of single channel microarray data. Bioinformatics. 2006; 22:897-899). AffylmGUI reads the raw Affymetrix CEL files directly, summarizes the gene expression values using RMA, and then uses LIMMA to identify statistically significant differences in gene expression (Smyth, Linear models and empirical bayes methods for assessing differential expression in microarray experiments. Stat Appl Genet Mol Biol. 2004; 3:Article3). LIMMA fits a linear model for every gene (like ANOVA or multiple regression analysis), and adjusts P values for multiple testings (Smyth, Linear models and empirical bayes methods for assessing differential expression in microarray experiments. Stat Appl Genet Mol Biol. 2004; 3:Article3). Differentially expressed genes were identified with a fold change >1.8.

To uncover enriched processes, data sets were analyzed by DAVID (Database for Annotation, Visualization and Integrated Discovery), a web-based tool that provides statistical methods for identifying over-represented biological themes and pathways within diverse and disparate gene lists (Dennis et al., DAVID: Database for Annotation, Visualization, and Integrated Discovery. Genome Biol. 2003; 4:3). DAVID also identifies over-represented biological themes in terms of their Gene Ontology (GO) terms and provides tools to visualize the distribution of genes on BioCarta and KFGG pathway maps. (Ashburner et al., Gene ontology: tool for the unification of biology. The Gene Ontology Consortium. Nat Genet. 2000; 25:25-29). GO provides consistent descriptions of genes in terms of biological processes and molecular function. Gene-enrichment analysis computes a modified Fisher exact p-value by comparing the ontological themes identified in our data set to total possible ontological processes present on the U133A chip. Ontological processes that had a p-value of less than 0.05 were selected.

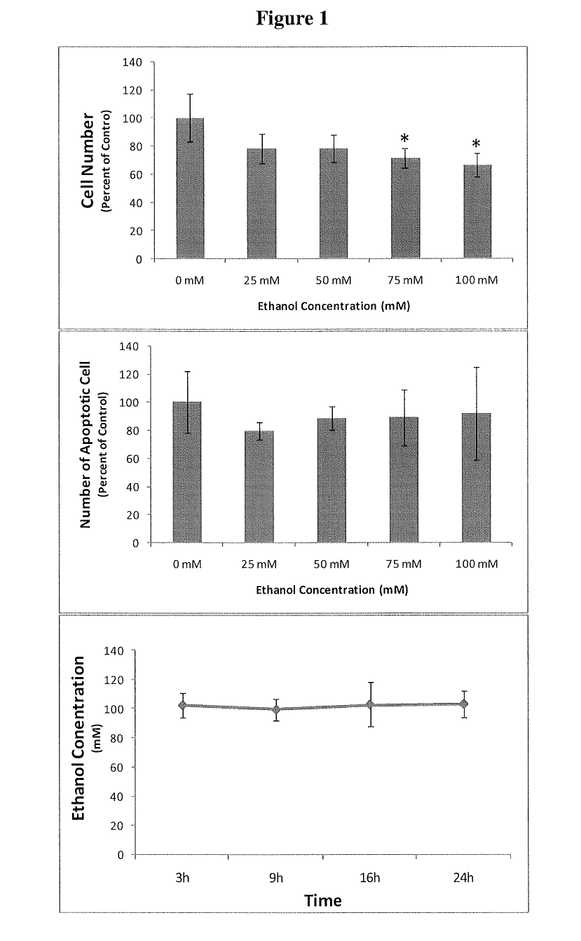

The effect of ethanol on growth and viability of AFSC. Exposure of AFSC to ethanol for 48 hours resulted in dose-dependent reduction in the rate of proliferation (FIG. 1A). The dose range for these studies was chosen to reflect physiologically relevant blood alcohol concentration (from the “legal” blood alcohol level to chronic alcoholics), as described above. The maximum concentration tested, 100 mM, caused a 33% reduction in the rate of proliferation whereas the 25 mM dose (equivalent to blood alcohol level of 0.12%), resulted in a 22% reduction in proliferation rate. Although cell numbers continued to increase in the presence of ethanol, this increase was partially inhibited by ethanol exposure. This result indicates that AFSC were growing in the presence of ethanol but at a slower rate. Thus, in all ethanol concentrations tested AFS cells continued to proliferate, but their proliferation was slower than cells grown in the absence of ethanol.

To determine if the ethanol-induced reduction in the rate of proliferation was due to cytotoxicity, we examined the effect of ethanol on cell viability by propidium iodide exclusion (FIG. 1B). AFSC that were not exposed to ethanol had a basal percentage of non-viable cells of 12.4%. The percentage of non-viable cells that were exposed to 25 mM to 100 mM ethanol ranged from 9.8 to 11.4%. These data suggest that ethanol did not have a significant effect on cell viability and that ethanol's reduction in the proliferation rate was not due to cytotoxicity. Concentrations of 100 mM ethanol and culture periods for 48 hours were used for the following experiments. Ethanol concentration present in the culture media did not change over a period of 24 hours (FIG. 1C).

The effect of ethanol on global gene expression. To characterize the differential response to ethanol, we performed large-scale transcriptome analysis on AFSC. AFSC were exposed to 100 mM ethanol for 48 hours in growth media, rather than a lineage specific differentiation medium, to prevent a bias toward identification of lineage-specific genes. To identify differentially expressed genes, we used Affymetrix GeneChips to generate datasets that were normalized and subjected to statistical analysis.

To uncover enriched processes, data sets were analyzed by DAVID, a web-based tool that identifies over-represented biological themes in a data set based on their Gene Ontology (GO) terms. GO provides consistent descriptions of genes in terms of biological processes and molecular function. We identified 65 genes that were up-regulated in response to ethanol and 16 genes that were down-regulated in response to ethanol.

These up-regulated and down-regulated genes were categorized by DAVID (database for annotation and visualization and integrated discovery), a web-based tool available from the National Institutes of Health, to identify enriched biological themes, particularly gene ontology terms. Categories are listed in Table 1A and 1B below (note that not every differentially expressed gene fell into one of the listed categories).

Processes that were identified in genes that were up-regulated in response to ethanol for 48 hours include skeletal development (5 genes) and ossification (5 genes), blood vessel development (4 genes), organ development (12 genes) and developmental processes (17 genes; Table 1) and were not present in the absence of ethanol. Genes in the skeletal development process include genes such as osteopontin, osteonectin, ectonucleotide pyrophosphatase, myocyte enhancer factor 2c and matrix metallopeptidase 14. Osteopontin and osteonectin are secreted phosphoproteins that are expressed at the early stage of osteogenic differentiation and have been shown to mediate cell-matrix interactions, cell adhesion, and differentiation (Butler, The nature and significance of osteopontin. Connect Tissue Res. 1989; 23:123-136; Delany et al., Osteonectin-null mutation compromises osteoblast formation, maturation, and survival. Endocrinology. 2003; 144:2588-2596; Strauss et al., Gene expression during osteogenic differentiation in mandibular condyles in vitro. J Cell Biol. 1990; 110:1369-1378). The identification of osteogenic genes that were up-regulated in response to ethanol suggests that ethanol exposure may predispose AFSC towards an osteogenic lineage.

Genes that were down-regulated in response to ethanol were also organized by their gene ontology. A predominate pathway identified in this dataset was embryonic development which includes genes encoding dickkopf homolog 1, neuregulin, and endoligen. Basic fibroblast growth factor/FGF2 was also down-regulated in response to ethanol (fold change of −2.0). bFGF is a potent mitogen and is an important factor in limb and neurogenesis (Fallon et al., FGF-2: apical ectodermal ridge growth signal for chick limb development. Science. 1994; 264:104-107; Raballo et al., Basic fibroblast growth factor (Fgf2) is necessary for cell proliferation and neurogenesis in the developing cerebral cortex. J. Neurosci. 2000; 20:5012-5023). The down-regulation of genes associated with these pathways suggests that ethanol restricts the range of differentiation potential of AFSC and may interfere with proper embryonic and fetal development.

TABLE 1A
Gene ontologies of up-regulated genes in response to ethanol

| Gene Category | | |
|---|---|---|
| Molecular function | Count | P- value |
| biomineral & ossification | 5 | 2.87E−04 |
| organ morphogenesis | 7 | 0.002 |
| organ development | 12 | 0.002 |
| reproductive developmental process | 4 | 0.0034 |
| skeletal development | 5 | 0.006 |
| system development | 13 | 0.009 |
| blood vessel morphogenesis | 4 | 0.0167 |
| tyrosine kinase signaling pathway | 4 | 0.0252 |
| IGF receptor signaling pathway | 2 | 0.0289 |
| developmental process | 17 | 0.05 |

TABLE 1B
Gene ontologies of down-regulated genes in response to ethanol

| Gene Category | | |
|---|---|---|
| Molecular function | Count | P- value |
| negative regulation of cellular process | 6 | 0.0024 |
| circulatory system process | 3 | 0.0098 |
| glucose transport | 2 | 0.0288 |
| embryonic development | 3 | 0.0295 |
| multi-organism process | 3 | 0.0334 |
| anatomical structure development | 6 | 0.036 |
| regulation of biological quality | 4 | 0.043 |

Table Legend: Genes were enriched based on the gene ontologies. Gene ontologies with a modified Fisher Exact P-value<0.05 were selected. Processes that were identified in genes that were up-regulated in response to ethanol include skeletal development, while genes that were down-regulated in response to ethanol included embryonic development.

TABLE 2A
Listing of genes up-regulated in response to ethanol*

signal sequence receptor gamma
lumican
solute carrier family 7 (cationic amino acid transporter, y+ system)
member 8
BCL2-associated X protein
regulator of G-protein signaling 2 24 kDa
calreticulin
ectonucleotide pyrophosphatase/phosphodiesterase 1
endothelin receptor type A
ring finger protein 128
chromosome 1 open reading frame 54
collagen type III alpha 1 (Ehlers-Danlos syndrome type IV, autosomal
dominant)
transducin (beta)-like 1X-linked
BCL2-associated X protein
secreted phosphoprotein 1 (osteopontin, bone sialoprotein I, early
T-lymphocyte activation 1)
nuclear receptor subfamily 2 group F member 2
SRY (sex determining region Y)-box 4
SRY (sex determining region Y)-box 11
collagen type III alpha 1 (Ehlers-Danlos syndrome type IV, autosomal
dominant)
ATPase H+ transporting lysosomal 70 kDa VI subunit A
cornichon homolog 3
DEAD (Asp-Glu-Ala-Asp) box polypeptide 17
malignant fibrous histiocytoma amplified sequence 1
UDP-N-acetyl-alpha-D-galactosamine
polypeptide N-acetylgalactosaminyltransferase 10 (GalNAc-T10)
chromosome 1 open reading frame 121
RNA binding motif protein 25
phospholipase A2 group IVA (cytosolic, calcium-dependent)
sphingomyelin phosphodiesterase acid-like 3A
SKI-like
KIAA1033
MADS box transcription enhancer factor 2 polypeptide C (myocyte
enhancer factor 2C)
ets variant gene 1
PTPRF interacting protein binding protein 1 (liprin beta 1)
GTP binding protein overexpressed in skeletal muscle
ATPase H+ transporting lysosomal 9 kDa V0 subunit e
SEC24 related gene family member D
plasminogen activator urokinase
chromosome 1 open reading frame 139
secreted protein acidic cysteine-rich (osteonectin)
SRY (sex determining region Y)-box 11
forkhead box F1
phosphoinositide-3-kinase regulatory subunit 1 (p85 alpha)
adaptor-related protein complex 1 sigma 1 subunit
insulin-like growth factor 1 receptor
transmembrane protein 35
iduronate 2-sulfatase (Hunter syndrome)
oxidation resistance 1
cyclin G2
degenerative spermatocyte homolog 1 lipid desaturase
ATPase Ca++ transporting plasma membrane 1
steroid-5-alpha-reductase alpha polypeptide 1 (3-oxo-5 alpha-steroid
delta 4-dehydrogenase alpha 1)
glycosyltransferase 8 domain containing 1
ADP-ribosylation factor-like 7
calumenin
low density lipoprotein-related protein 12
matrix metallopeptidase 14
3-hydroxyisobutyryl-Coenzyme A hydrolase
*Note that there is some redundancy in the genes listed above. Listing of a gene more than once means that there were two sequences that represent the same gene that was identified as upregulated.

TABLE 2B
Listing of genes down-regulated in response to ethanol

H2A histone family member X
ubiquitin-conjugating enzyme E2I
heterogeneous nuclear ribonucleoprotein A3
dickkopf homolog 1
glutathione peroxidase 3
endothelin 1
pentraxin-related gene
ankyrin repeat domain 1
TSPY-like 4
fibroblast growth factor 2 (basic)
microfibrillar associated protein 5
heterogeneous nuclear ribonucleoprotein H1 (H)
deleted in liver cancer 1
ADAM metallopeptidase with thrombospondin type 1 motif, 1
oxytocin receptor
neuregulin 1

To identify alcohol-related changes in gene expression, global gene expression analysis was performed after 48 hours of exposure to ethanol. This duration was chosen in order to expose AFSC throughout the cell cycle (36 hours for AFSC) and to identify the late response (not early oxidative stress) of AFSC to ethanol. Ethanol-responsive genes were analyzed according to their gene ontology and revealed unique pathways that pertain to bone development. Accordingly, our functional analysis of osteogenic differentiation suggests that ethanol's effect on gene expression early in differentiation predisposes AFSC into an osteogenic lineage, and subsequent increases in alkaline phosphatase and calcium deposition provide a potential mechanism of ethanol on osteogenic differentiation. Premature differentiation of stem cells can deplete the stem cell population, resulting in fewer number of cells which may explain some of the clinical features of FASD such a short stature and craniofacial malformations.

### Example 2

### Osteogenic Induction of AFSC Exposed to Ethanol

A simplified osteogenic differentiation paradigm was utilized to examine the potential mechanism of ethanol on the progression of stem cells into an osteogenic lineage.

Human AFSC were induced to differentiate into osteogenic cell types as described previously (De Coppi et al., Isolation of amniotic stem cell lines with potential for therapy. Nat Biotechnol. 2007; 25:100-106). Briefly, AFSC was cultured in DMEM low glucose with 10% FBS supplemented with 100 nM dexamethasone (Sigma-Aldrich), 10 mM beta-glycerophosphate (Sigma-Aldrich) and 0.05 mM ascorbic acid-2-phosphate (Wako Chemicals, Irving, Tex.). Cells were grown to confluency and then treated with ethanol for the first 48 hours of osteogenic differentiation to control for ethanol's antiproliferative effect. Cell number was determined after 24 and 48 hours of ethanol and bone differentiation. Ethanol had no effect on cell number when cells were grown to confluency (data not shown).

As described in De Coppi et al., AFSC develop into osteoblast-like morphology within 1 week of differentiation and by sixteen days will form bone-like lamellar structures. Furthermore, they express mRNA and protein for alkaline phosphatase after one week of osteogenic differentiation. Functional assays for calcium deposition show strong histological staining by alizarin red. They also deposit calcium, show strong histochemical staining for alkaline phosphatase and secrete this enzyme (De Coppi et al., Isolation of amniotic stem cell lines with potential for therapy. Nat Biotechnol. 2007; 25:100-106). Bone differentiation was analyzed by mRNA expression of osteopontin and bone-specific alkaline phosphatase, alkaline phosphatase activity, and stained with Alizarin Red to quantify extracellular calcium deposition at Day 23 of differentiation.

To analyze the functional properties of osteogenic differentiation, the presence of calcium in cell culture was determined by alizarin red (Sigma) staining at day 23 of osteogenic differentiation. Cells were fixed with 4% formaldehyde for 15 min. Fixed cells were incubated with 0.5% alizarin red solution in water and pH adjusted to 4.0 for 1 minute. Cells were then washed three times with deionized water and once with 70% ethanol then allowed to dry. Calcium deposition was quantified by extracting alizarin red stain with 100 mM cetylpyridinium chloride (Sigma) at room temperature for three hours. The absorbance of the extracted alizarin red stain was measured at 540 nm. The concentration of alizarin red staining in the samples was determined by comparing the absorbance values with those obtained from an alizarin red standard curve.

Alkaline phosphatase activity was measured using p-nitrophenyl phosphate liquid substrate system (Sigma). Cells grown in 24-well plates were rinsed in PBS and incubated with 0.15% Triton X-100 for 30 mins. Two hundred uL of p-nitrophenyl phosphate solution was added to the Triton-X 100 solution. Cells were incubated in the dark for 1 hour and read spectrophotometrically at 405 nm.

Results are expressed as mean±S.D. for quantitative data. Analysis of Variance (ANOVA) was used to identify statistically significant differences between groups. Alternatively, two-tailed tests of significance was computed to determine relationships between ethanol-treated and control groups. Statistical significance was set at p<0.05.

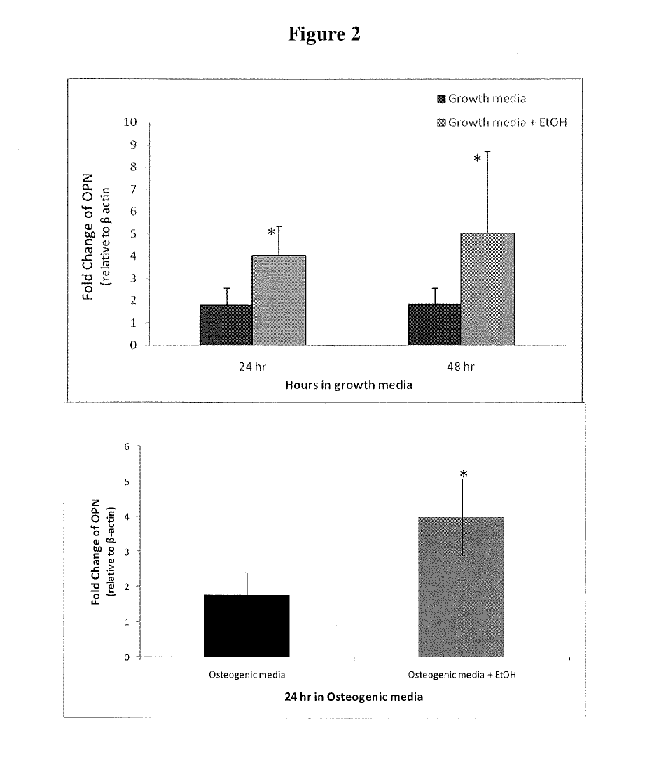

The effect of ethanol on osteopontin expression. Osteopontin is a phosphorylated glycoprotein that is secreted at an early stage of osteogenic differentiation. It is abundant in mineralized tissue and may be implicated in bone formation (Butler, The nature and significance of osteopontin. Connect Tissue Res. 1989; 23:123-136; Strauss et al., Gene expression during osteogenic differentiation in mandibular condyles in vitro. J Cell Biol. 1990; 110:1369-1378; Kojima et al., In vitro and in vivo effects of the overexpression of osteopontin on osteoblast differentiation using a recombinant adenoviral vector. J. Biochem. 2004; 136:377-386). To further define the effect of ethanol on osteopontin expression, real time-PCR was performed after 24 and 48 hours of exposure to 100 mM ethanol. Although control AFSC expressed osteopontin, ethanol-exposed AFSC showed an increase in mRNA expression of osteopontin (FIG. 2A). After 24 hours of ethanol exposure, the expression of osteopontin increased by 2.2-fold. Exposure to ethanol for 48 hours increased the expression of osteopontin by a fold change of 2.8. The increase in osteopontin expression was significant at 24 and 48 hours (p<0.02 and p<0.036). These results indicate that ethanol increases the mRNA expression of osteopontin in AFSC after 24 hours and is further maintained after 48 hours. Thus, when AFSC were exposed to ethanol in growth media, the ethanol-induced increase in osteopontin expression may push AFSC towards an osteogenic lineage.

The previous experiment showed that ethanol exposure increases osteopontin expression when cultured in growth media. It was next determined if ethanol has a similar effect when cells are exposed to osteogenic differentiation media. The conditions used for the in vitro induction of osteogenic differentiation of AFSC include: 1) ascorbic acid, which is essential for the differentiation and function of osteoblasts (Bellows et al., Mineralized bone nodules formed in vitro from enzymatically released rat calvaria cell populations. Calcif Tissue Int. 1986; 38:143-154) and is required for collagen synthesis (Murad et al., Regulation of collagen synthesis by ascorbic acid. Proc Natl Acad Sci USA. 1981; 78:2879-2882); 2) beta-glycerol phosphate, which provides an organic phosphate for the formation of hydroxyapatite (Bellows et al., Mineralized bone nodules formed in vitro from enzymatically released rat calvaria cell populations. Calcif Tissue Int. 1986; 38:143-154; Bellows et al., Inorganic phosphate added exogenously or released from beta-glycerophosphate initiates mineralization of osteoid nodules in vitro. Bone Miner. 1992; 17:15-29); and 3) dexamethasone, a glucocorticoid that induces transcription at the promoter of osteogenic genes (Ogata et al., Glucocorticoid regulation of bone sialoprotein (BSP) gene expression. Identification of a glucocorticoid response element in the bone sialoprotein gene promoter. Eur J. Biochem. 1995; 230:183-192). However, these additives have other functions that are not limited to osteogenic differentiation. Dexamethasone is used to induce adipogenic differentiation in AFSC (De Coppi et al., Isolation of amniotic stem cell lines with potential for therapy. Nat Biotechnol. 2007; 25:100-106). Ascorbic acid is also an antioxidant and may negate the effects of ethanol in osteogenic media.

To determine whether ethanol interferes with the action of these additives, we examined the mRNA expression of osteopontin after 24 hours of 100 mM ethanol in osteogenic media. Ethanol increased the expression of osteopontin by 2-fold in the presence of osteogenic media (FIG. 2B). These experiments suggest that ethanol induces osteopontin expression in both uncommitted AFSC and AFSC-committed towards an osteogenic cell type.

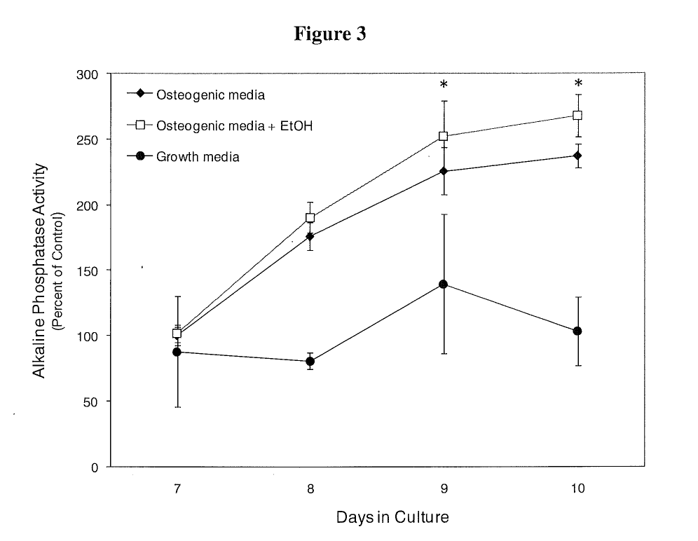

The effect of ethanol on alkaline phosphatase activity. We further tested the effect of ethanol on alkaline phosphatase activity, which is an established marker of osteoblasts (Bellows et al., Initiation and progression of mineralization of bone nodules formed in vitro: the role of alkaline phosphatase and organic phosphate. Bone Miner. 1991; 14:27-40; Fedde et al., Alkaline phosphatase knock-out mice recapitulate the metabolic and skeletal defects of infantile hypophosphatasia. J Bone Miner Res. 1999; 14:2015-2026). Our previous study showed that AFSC begin to express alkaline phosphatase activity after 8 days of osteogenic differentiation. To address the question of whether prior ethanol exposure has an effect on the alkaline phosphatase activity, AFSC were exposed to 100 mM ethanol for the first 48 hours in osteogenic media, continued to differentiate and assayed for alkaline phosphatase activity at various time points (FIG. 3). After 8 days of osteogenic differentiation, alkaline phosphatase activity rose above control levels and continued to increase until day 10 of osteogenic differentiation. However, ethanol exposure during the first 48 hours showed a small but significant increase in alkaline phosphatase activity at day 9 and 10. AFSC that were not exposed to osteogenic media expressed a basal level of alkaline phosphatase activity. These results suggest that ethanol has a persistent and enhancing effect on differentiation that can be seen days beyond the ethanol exposure period.

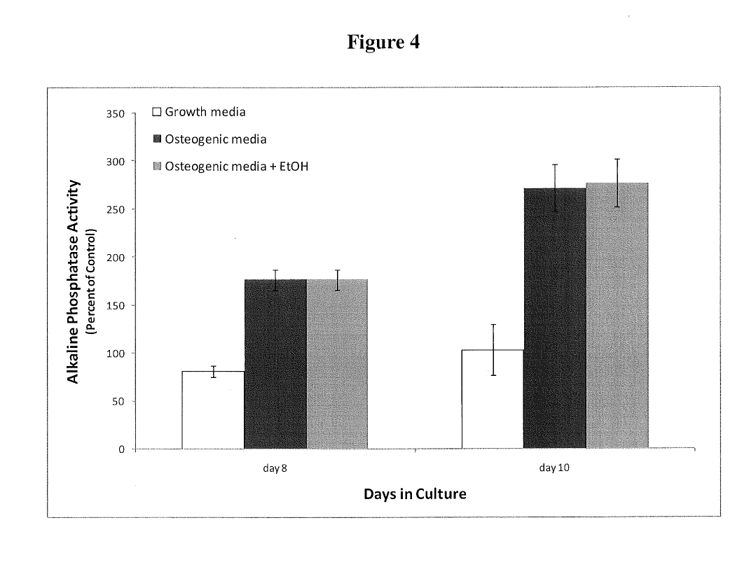

In order to test if ethanol may have different effects depending on the stage of differentiation, we cultured AFSC in osteogenic media for 8 days without ethanol. We then treated AFSC with or without 100 mM of ethanol on day 8 for 48 hours. We exposed AFSC to ethanol at this stage of osteogenic differentiation in order to determine whether ethanol has a direct effect on alkaline phosphatase activity. AFSC exposed to 100 mM of ethanol at day 8 of osteogenic differentiation for 48 hours had no statistically significant effect on alkaline phosphatase activity (FIG. 4). Collectively, these experiments suggest that ethanol exposure only early in differentiation has a significant effect on alkaline phosphatase compared to exposure during the midpoint of differentiation.

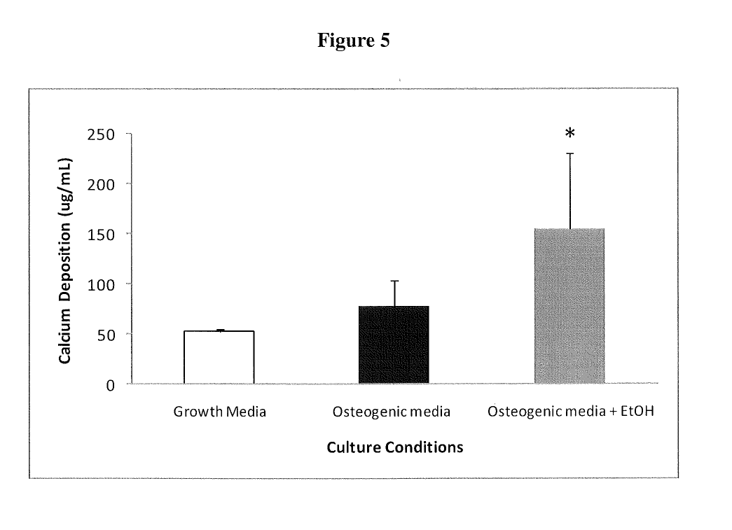

The effect of ethanol on calcium deposition. Because transient ethanol exposure increased the expression and activation of genes involved with mineralization, we sought to determine the effect of ethanol on calcium deposition. Cells were exposed to osteogenic media with or without 100 mM of ethanol for 48 hours. Ethanol was removed and the AFSC were allowed to terminally differentiate. At day 23, AFSC were measured for calcium deposition by histological staining. The effects of ethanol treatment on calcium deposition are shown in FIG. 5. AFSC exposed to ethanol during the first 48 hours of osteogenic differentiation produced 155.06±75.85 μg/mL of calcium while non-exposed AFSC produced 77.40±26.85 μg/mL of calcium. Calcium deposition was detected in non-differentiated AFSC at a basal level of 52.5±2.223 μg/ml. These data suggest that the effect of transient ethanol exposure on osteogenic genes during early differentiation is directly correlated with the AFSCs' ability to deposit calcium.

Studies suggest that the window of enhanced susceptibility of the fetus to ethanol occurs during the first trimester, which is a period of organ development (Becker et al., Teratogenic actions of ethanol in the mouse: a minireview. Pharmacol Biochem Behay. 1996; 55:501-513). To determine whether ethanol's effect depends on the stage of differentiation, we exposed cells to ethanol at a midpoint of differentiation and measured alkaline phosphatase activity. Ethanol exposure during the midpoint of differentiation did not have an effect on alkaline phosphatase activity.

The results suggest that if AFSC were exposed to ethanol prior to the expression of alkaline phosphatase activity, there was an effect. However, if cells were exposed to ethanol when alkaline phosphatase is expressed, ethanol does not have an effect.

The effects of ethanol on a limited variety of stem cells have previously been reported, with some studies showing enhanced and some showing reduced differentiation potential. Neural stem (NSCs) and progenitor cells have been the most commonly studied. Ethanol has been shown to alter the differentiation potential of NSCs by enhancing astrocytic and oligodendrocytic differentiation and decreasing neuronal differentiation (Tateno, Ukai et al., 2005). Adult bone-marrow derived stem cells (BMSCs) have also been employed and, as opposed to the current study, inhibition of osteogenic differentiation has been shown (Gong and Wezeman, 2004). Another study, which used immortalized human fetal osteoblasts to examine the effect of ethanol on skeletal development by analysis of osteogenic gene expression (Maran, Zhang et al., 2001) demonstrated little or no effect of ethanol. As for the BMSCs, the response of these immortalized fetal osteoblasts may not reflect that of prenatal stem cells.

Ethanol has been shown to enhance cartilage differentiation in embryonic limb mesenchyme cultures (Kulyk and Hoffman, 1996; Shukla, Velazquez et al., 2008). While these cells seem to be lineage restricted because they spontaneously differentiate into chondrocytes, naive AFSCs are not lineage restricted to osteogenesis. Thus, ethanol may act to lineage restrict AFSCs to osteogenesis by elevating the expression of osteogenesis-specific genes.

We have previously noted that AFSCs express RUNX2 and osteocalcin during osteogenic differentiation (De Coppi, Bartsch et al., 2007). Although these genes may be more specific as osteogenic markers than ALP and osteopontin, RUNX2 expression, measured by real-time PCR, was not changed after 48 hours of ethanol exposure.

### Example 3

### Testing of AFSC for Prenatal Alcohol Exposure

Plate two mL of amniotic fluid samples on 6-well petri dishes and expand the cells. When cells reach a confluency of 40,000 immunoselect amniotic fluid cells by c-kit to obtain amniotic fluid stem cells (AFSC). Grow cells until a line is established, typically 1 week after immunoselection. One or more of the following four tests are then performed, each at a cell density of approximately 6,500 cells/cm².

Control AFSC may be prepared in like manner from a non-ethanol exposed donor or an established non-ethanol exposed AFSC line. Other control cells and comparisons thereto may be determined through routine testing by those of skill in the art following the guidance provided herein.

Test 1: Determine gene expression by microarray as described above.

(a): Upregulation of osteogenic genes as compared to fibroblast cells and/or control AFSC indicates alcohol exposure.

(b): Compared to control AFSC, ethanol exposed AFSC show a relative 2-fold change in gene expression of particular genes listed above in Table 2A and Table 2B and/or in categories listed above in Table 1A and Table 1B (upregulated and down-regulated, respectively).

Test 2: Measure osteopontin (spp1) expression. A two-fold upregulation of spp1 as compared to control AFCS not exposed to ethanol indicates alcohol exposure.

Test 3. Differentiate AFSC with osteogenic induction. Measure alkaline phosphatase expression at day 8, 10, and 12 of osteogenic differentiation. Activity of alkaline phosphatase above a threshold of 6,000 Units/L indicates alcohol exposure.

Test 4. Measure calcium deposition at day 23 after differentiation of AFSC with osteogenic induction. Calcium deposition above a threshold of 155 μg/mL at day 23 of differentiation indicates alcohol exposure.

The foregoing is illustrative of the present invention, and is not to be construed as limiting thereof. The invention is defined by the following claims, with equivalents of the claims to be included therein.

That which is claimed is:

1. A method for determining ethanol exposure of a prenatal subject comprising:
providing amniotic fluid stem cells collected from amniotic fluid surrounding said prenatal subject; and
measuring whether or not said amniotic fluid stem cells have a two-fold or greater upregulation of expression of each gene of a first predetermined combination of genes as compared to expression of each gene of said first predetermined combination by control amniotic fluid stem cells,
wherein said two-fold or greater upregulation of expression of each gene of said first predetermined combination indicates ethanol exposure of said prenatal subject.

2. The method of claim 1, wherein said first predetermined combination of genes comprises one or more genes selected from the group consisting of: signal sequence receptor gamma; lumican; solute carrier family 7 (cationic amino acid transporter, y+ system) member 8; BCL2-associated X protein; regulator of G-protein signaling 2 24 kDa; calreticulin; ectonucleotide pyrophosphatase/phosphodiesterase 1; endothelin receptor type A; ring finger protein 128; chromosome 1 open reading frame 54; collagen type III alpha 1 (Ehlers-Danlos syndrome type IV, autosomal dominant); transducin (beta)-like 1×-linked; BCL2-associated X protein; secreted phosphoprotein 1 (osteopontin, bone sialoprotein I, early T-lymphocyte activation 1); nuclear receptor subfamily 2 group F member 2; SRY (sex determining region Y)-box 4; SRY (sex determining region Y)-box 11; collagen type III alpha 1 (Ehlers-Danlos syndrome type IV, autosomal dominant); ATPase H+ transporting lysosomal 70 kDa V1 subunit A; cornichon homolog 3; DEAD (Asp-Glu-Ala-Asp) box polypeptide 17; malignant fibrous histiocytoma amplified sequence 1; UDP-N-acetyl-alpha-D-galactosamine; polypeptide N-acetylgalactosaminyltransferase 10 (GalNAc-T10); chromosome 1 open reading frame 121; RNA binding motif protein 25; phospholipase A2 group IVA (cytosolic, calcium-dependent); sphingomyelin phosphodiesterase acid-like 3A; SKI-like; KIAA1033; MADS box transcription enhancer factor 2 polypeptide C (myocyte enhancer factor 2C); ets variant gene 1; PTPRF interacting protein binding protein 1 (liprin beta 1); GTP binding protein overexpressed in skeletal muscle; ATPase H+ transporting lysosomal 9 kDa V0 subunit e; SEC24 related gene family member D; plasminogen activator urokinase; chromosome 1 open reading frame 139; secreted protein acidic cysteine-rich (osteonectin); SRY (sex determining region Y)-box 11; forkhead box F1; phosphoinositide-3-kinase regulatory subunit 1 (p85 alpha); adaptor-related protein complex 1 sigma 1 subunit; insulin-like growth factor 1 receptor; transmembrane protein 35; iduronate 2-sulfatase (Hunter syndrome); oxidation resistance 1; cyclin G2; degenerative spermatocyte homolog 1 lipid desaturase; ATPase Ca++transporting plasma membrane 1; steroid-5-alpha-reductase alpha polypeptide 1 (3-oxo-5 alpha-steroid delta 4-dehydrogenase alpha 1); glycosyltransferase 8 domain containing 1; ADP-ribosylation factor-like 7; calumenin; low density lipoprotein-related protein 12; matrix metallopeptidase 14; and 3-hydroxyisobutyryl-Coenzyme A hydrolase.

3. The method of claim 2, wherein said first predetermined combination of genes comprises five or more genes selected from said group.

4. The method of claim 2, wherein said first predetermined combination of genes comprises 10 or more genes selected from said group.

5. The method of claim 2, wherein said first predetermined combination of genes comprises 20 or more genes selected from said group.

6. The method of claim 1, further comprising:
measuring whether or not said amniotic fluid stem cells have a two-fold or greater down-regulation of expression of each gene of a second predetermined combination of genes as compared to expression of each gene of said second predetermined combination by control amniotic fluid stem cells,
wherein said two-fold or greater downregulation of expression of each gene of said second predetermined combination indicates ethanol exposure of said prenatal subject.

7. The method of claim 6, wherein said second predetermined combination of genes comprises one or more genes selected from the group consisting of: H2A histone family member X; ubiquitin-conjugating enzyme E2I; heterogeneous nuclear ribonucleoprotein A3; dickkopf homolog 1; glutathione peroxidase 3; endothelin 1; pentraxin-related gene; ankyrin repeat domain 1; TSPY-like 4; fibroblast growth factor 2 (basic); microfibrillar associated protein 5; heterogeneous nuclear ribonucleoprotein H1 (H); deleted in liver cancer 1; ADAM metallopeptidase with thrombospondin type 1 motif, 1; oxytocin receptor; and neuregulin 1.

8. The method of claim 6, wherein said second predetermined combination of genes comprises at least five genes selected from the group consisting of: H2A histone family member X; ubiquitin-conjugating enzyme E21; heterogeneous nuclear ribonucleoprotein A3; dickkopf homolog 1; glutathione peroxidase 3; endothelin 1; pentraxin-related gene; ankyrin repeat domain 1; TSPY-like 4; fibroblast growth factor 2 (basic); microfibrillar associated protein 5; heterogeneous nuclear ribonucleoprotein H1 (H); deleted in liver cancer 1; ADAM metallopeptidase with thrombospondin type 1 motif, 1; oxytocin receptor; and neuregulin 1.

9. The method of claim 6, wherein said second predetermined combination of genes comprises at least 10 genes selected from the group consisting of: H2A histone family member X; ubiquitin-conjugating enzyme E21; heterogeneous nuclear ribonucleoprotein A3; dickkopf homolog 1; glutathione peroxidase 3; endothelin 1; pentraxin-related gene; ankyrin repeat domain 1; TSPY-like 4; fibroblast growth factor 2 (basic); microfibrillar associated protein 5; heterogeneous nuclear ribonucleoprotein H1 (H); deleted in liver cancer 1; ADAM metallopeptidase with thrombospondin type 1 motif, 1; oxytocin receptor; and neuregulin 1.

10. The method of claim 1, wherein said prenatal subject is human.

11. The method of claim 10, wherein said amniotic fluid stem cells are collected between 8 and 22 weeks of gestation.

12. The method of claim 1, wherein said measuring comprises nucleic acid amplification.

13. The method of claim 1, wherein said measuring comprises analysis of a microarray comprising said first predetermined combination.

14. The method of claim 1, wherein said measuring comprises analysis of a microarray consisting essentially of said first predetermined combination.

15. A combination consisting essentially of a plurality of cDNAs encoding at least five genes selected from the group consisting of: signal sequence receptor gamma; lumican; solute carrier family 7 (cationic amino acid transporter, y+ system) member 8; BCL2-associated X protein; regulator of G-protein signaling 2 24 kDa; calreticulin; ectonucleotide pyrophosphatase/phosphodiesterase 1; endothelin receptor type A; ring finger protein 128; chromosome 1 open reading frame 54; collagen type III alpha 1 (Ehlers-Danlos syndrome type IV, autosomal dominant); transducin (beta)-like 1×-linked; BCL2-associated X protein; secreted phosphoprotein 1 (osteopontin, bone sialoprotein I, early T-lymphocyte activation 1); nuclear receptor subfamily 2 group F member 2; SRY (sex determining region Y)-box 4; SRY (sex determining region Y)-box 11; collagen type III alpha 1 (Ehlers-Danlos syndrome type IV, autosomal dominant); ATPase H+ transporting lysosomal 70 kDa V1 subunit A; cornichon homolog 3; DEAD (Asp-Glu-Ala-Asp) box polypeptide 17; malignant fibrous histiocytoma amplified sequence 1; UDP-N-acetyl-alpha-D-galactosamine; polypeptide N-acetylgalactosaminyltransferase 10 (GalNAc-T10); chromosome 1 open reading frame 121; RNA binding motif protein 25; phospholipase A2 group IVA (cytosolic, calcium-dependent); sphingomyelin phosphodiesterase acid-like 3A; SKI-like; KIAA1033; MADS box transcription enhancer factor 2 polypeptide C (myocyte enhancer factor 2C); ets variant gene 1; PTPRF interacting protein binding protein 1 (liprin beta 1); GTP binding protein overexpressed in skeletal muscle; ATPase H+ transporting lysosomal 9 kDa V0 subunit e; SEC24 related gene family member D; plasminogen activator urokinase; chromosome 1 open reading frame 139; secreted protein acidic cysteine-rich (osteonectin); SRY (sex determining region Y)-box 11; forkhead box F1; phosphoinositide-3-kinase regulatory subunit 1 (p85 alpha); adaptor-related protein complex 1 sigma 1 subunit; insulin-like growth factor 1 receptor; transmembrane protein 35; iduronate 2-sulfatase (Hunter syndrome); oxidation resistance 1; cyclin G2; degenerative spermatocyte homolog 1 lipid desaturase; ATPase Ca++ transporting plasma membrane 1; steroid-5-alpha-reductase alpha polypeptide 1 (3-oxo-5 alpha-steroid delta 4-dehydrogenase alpha 1); glycosyltransferase 8 domain containing 1; ADP-ribosylation factor-like 7; calumenin; low density lipoprotein-related protein 12; matrix metallopeptidase 14; and 3-hydroxyisobutyryl-Coenzyme A hydrolase.

16. The combination of claim 15, wherein said plurality of cDNAs encodes at least ten genes selected from said group.

17. The combination of claim 15, wherein said plurality of cDNAs encodes at least 20 genes selected from said group.

18. The combination of claim 15, wherein said cDNAs are immobilized on a substrate.

19. A method for determining ethanol exposure of a prenatal subject comprising:
providing amniotic fluid stem cells collected from amniotic fluid surrounding said prenatal subject; and
measuring whether or not said amniotic fluid stem cells have a two-fold or greater upregulation of expression of secreted phosphoprotein 1 (osteopontin, bone sialoprotein I, early T-lymphocyte activation 1) as compared to expression of secreted phosphoprotein 1 (osteopontin, bone sialoprotein I, early T-lymphocyte activation 1) by control amniotic fluid stem cells or fibroblast cells,
wherein said two-fold or greater upregulation of expression of secreted phosphoprotein 1 (osteopontin, bone sialoprotein I, early T-lymphocyte activation 1) indicates ethanol exposure of said prenatal subject.

20. The method of claim 19, wherein said prenatal subject is human.

21. The method of claim 20, wherein said amniotic fluid stem cells are collected between 8 and 22 weeks of gestation.

22. The method of claim 19, wherein said measuring comprises nucleic acid amplification.

23. A method for determining ethanol exposure of a prenatal subject comprising:
providing amniotic fluid stem cells collected from amniotic fluid surrounding said prenatal subject; and
measuring whether or not said amniotic fluid stem cells have a two-fold or greater down-regulation of expression of each gene of a predetermined combination of genes as compared to expression of each gene of said predetermined combination by control amniotic fluid stem cells or fibroblast cells,
wherein said two-fold or greater downregulation of expression of each gene of said predetermined combination indicates ethanol exposure of said prenatal subject.

24. The method of claim 23, wherein said predetermined combination of genes comprises one or more genes selected from the group consisting of: H2A histone family member X; ubiquitin-conjugating enzyme E21; heterogeneous nuclear ribonucleoprotein A3; dickkopf homolog 1; glutathione peroxidase 3; endothelin 1; pentraxin-related gene; ankyrin repeat domain 1; TSPY-like 4; fibroblast growth factor 2 (basic); microfibrillar associated protein 5; heterogeneous nuclear ribonucleoprotein H1 (H); deleted in liver cancer 1; ADAM metallopeptidase with thrombospondin type 1 motif, 1; oxytocin receptor; and neuregulin 1.

25. The method of claim 23, wherein said predetermined combination of genes comprises at least five genes selected from said group.

26. The method of claim 23, wherein said predetermined combination of genes comprises at least 10 genes selected from said group.

27. The method of claim 23, wherein said prenatal subject is human.

28. The method of claim 27, wherein said amniotic fluid stem cells are collected between 8 and 22 weeks of gestation.

29. The method of claim 23, wherein said measuring comprises nucleic acid amplification.

30. The method of claim 23, wherein said measuring comprises analysis of a microarray comprising said predetermined combination.

31. The method of claim 23, wherein said measuring comprises analysis of a microarray consisting essentially of said predetermined combination.

32. A combination consisting essentially of a plurality of cDNAs encoding at least five genes selected from the group consisting of: H2A histone family member X; ubiquitin-conjugating enzyme E21; heterogeneous nuclear ribonucleoprotein A3; dickkopf homolog 1; glutathione peroxidase 3; endothelin 1; pentraxin-related gene; ankyrin repeat domain 1; TSPY-like 4; fibroblast growth factor 2 (basic); microfibrillar associated protein 5; heterogeneous nuclear ribonucleoprotein H1 (H); deleted in liver cancer 1; ADAM metallopeptidase with thrombospondin type 1 motif, 1; oxytocin receptor; and neuregulin 1.

33. The combination of claim 32, wherein said plurality of cDNAs encodes at least 10 genes selected from said group.

34. A method for determining ethanol exposure of a prenatal subject comprising:
providing amniotic fluid stem cells collected from amniotic fluid surrounding said prenatal subject;
differentiating said amniotic fluid stem cells in osteogenic medium; and
measuring whether or not said amniotic fluid stem cells have an alkaline phosphatase activity above a threshold of 6,000 Units/L at day 8, 10, 11 or 12 of said differentiating, wherein said alkaline phosphatase activity is measured as Units/L=liberation of 1 mmol of PNP per minute at 37° C. incubation per liter,
wherein alkaline phosphatase activity above a threshold of 6,000 Units/L at day 8, 10, 11 or 12 indicates ethanol exposure of said prenatal subject.

35. The method of claim 34, further comprising:
measuring whether or not calcium deposition at day 23 after said differentiating is above a threshold of 155 μg/mL,
wherein calcium deposition above a threshold of 155 μg/mL at day 23 of said differentiating indicates ethanol exposure of said prenatal subject.

36. The method of claim 34, wherein said prenatal subject is human.

37. The method of claim 36, wherein said amniotic fluid stem cells are collected between 8 and 22 weeks of gestation.

38. A method for determining ethanol exposure of a prenatal subject comprising:
providing amniotic fluid stem cells collected from amniotic fluid surrounding said prenatal subject;
differentiating said amniotic fluid stem cells in osteogenic medium; and
measuring whether or not calcium deposition at day 23 after said differentiating is above a threshold of 155 μg/mL,
wherein calcium deposition above a threshold of 155 μg/mL at day 23 of said differentiating indicates ethanol exposure of said prenatal subject.

39. The method of claim 38, wherein said prenatal subject is human.

40. The method of claim 39, wherein said amniotic fluid stem cells are collected between 8 and 22 weeks of gestation.
